(19) United States
(12) Patent Application Publication
(10) Pub. No.: US 2001/0028894 A1
(43) Pub. Date: Oct. 11, 2001

(54) Composite structure having an adhesive matrix containing one or more active agents

(76) Inventor: Jean-Louis Gueret, Paris (FR)

Correspondence Address: OPPENHEIMER WOLFF & DONNELLY LLP, Suite 3800, 2029 Century Park East, Los Angeles, CA, 90067-3028, US

(21) Appl. No.: 09/779,095

(22) Filed: Feb. 8, 2001

(30) Foreign Application Priority Data
Feb. 16, 2000 (FR) 00 01903

(51) Int. Cl. A61K 9/70

(52) U.S. Cl. 424/443; 442/123

(57) ABSTRACT

A composite structure such as a patch, a disk, or a towelette for use in particular in treating, making up, or cleaning the skin or the hair. The structure comprises at least one adhesive matrix based on a permanent adhesive and present between two support layers, at least one of which is permeable to a solvent, the two support layers being permanently bonded to the adhesive matrix, the adhesive matrix containing at least one active agent soluble in said solvent and possibly a filler, the nature and the quantity of the active agent(s) and/or the filler being selected to make it possible, when the composite structure is wetted by the solvent, for the active agent(s) to be released and to diffuse towards the surface to be treated.

The present invention relates to composite structures such as patches, disks, or towelettes used for treating, making up, or cleaning the skin or the hair, and comprising at least a support layer coated in an adhesive matrix and one or more active agents.

### BACKGROUND OF THE INVENTION

There exists a need to improve the conservation of the active agent(s) contained in such composite structures.

There also exists a need to facilitate manufacture of a range of composite structures having different combinations of active agents, or indeed containing active agents that should be put into contact only extemporaneously.

There also exists a need to have composite structures offering different possibilities of use, for example making it possible to release a selected one out of two active agents or to release two agents, one after the other, or to clean the skin and then apply a predetermined active agent to the skin cleaned in this way.

### OBJECTS AND SUMMARY OF THE INVENTION

An object of the present invention is to provide a novel composite structure for impregnating at the time of use with a solvent, e.g. water, a lotion, or an oil, and for applying to the skin or the hair, and also satisfying all or some of the needs listed above.

This composite structure comprises at least one adhesive matrix based on a permanent adhesive and present between two support layers, at least one of which is permeable to a solvent, the two support layers being permanently bonded to the adhesive matrix, the adhesive matrix containing at least one active agent soluble in said solvent and possibly a filler, the nature and the quantity of the active agent(s) and/or the filler being selected to make it possible, when the composite structure is wetted by the solvent, for the active agent(s) to be released and to diffuse towards the surface to be treated.

In a particular embodiment, the adhesive matrix contains one or more active agents soluble in the solvent and/or capable of swelling on coming into contact with said solvent, and in sufficient quantity for the matrix to lose its cohesion on contact with the solvent and to release the active agent(s).

Still in a particular embodiment, as a variant or in addition, the adhesive matrix contains a filler of one or more compounds capable of swelling on contact with the solvent, in sufficient quantity for the matrix to lose its cohesion on contact with the solvent and to release the active agent(s).

Still in a particular embodiment, as a variant or in addition, the adhesive matrix contains a filler of one or more substantially inert compounds in sufficient quantity for the matrix to lose its cohesion on contact with the solvent and to release the active agent(s).

The solvent in question can be water.

The composite structure of the invention makes it easy to package one or more active agents by incorporating them in one or more adhesive matrices.

Conservation takes place in the anhydrous state under good conditions since the composite structure need not be impregnated with water or lotion until the time of use.

This makes it possible to avoid using preservatives or to reduce the concentration thereof.

Advantageously, the adhesive matrix contains one or more moisture-absorbing compounds and preferably contains 0.2% to 60% by weight of a moisture-absorbing compound, preferably 0.5% to 40%, with the compound being selected, for example, from polyacrylates, silicas, cotton fibers, starches, alginates, calcium or magnesium carbonates, viscose, cellulose, and freeze-dried substances, this list not being limiting.

In addition to moisture-absorbing compounds, the adhesive matrix advantageously includes one or more substances capable of lowering its adhesive power and enabling it to burst on coming into contact with the solvent so as to facilitate release of the active agent(s).

Of such substances, particular mention can be made, for example, of substances that are substantially inert such as microbeads or a powder of an inert compound, e.g. the polyamide powder known under the name ORGASOL.

The adhesive matrix can include one or more active agents selected from the following list: vitamin C; vitamin A; vitamin F; glycerin; laponite; wetting agents; collagen; salicylic acid; tic acid; caffeine; aromatic essential oils; coloring agents; anti-oxidants; free radical scavengers; moisturizers; depigmenting agents; liporegulators; anti-acne agents; antidandruff agents; anti-aging agents; softeners; antiwrinkle agents; keratolitic agents; anti-inflammatory agents; fresheners; healing agents; vascular protectors; antibacterial agents; antifungal agents; antiperspirants; deodorants; skin conditioners; anesthetics; immunomodulators; and nourishing agents, this list naturally not being limiting.

The adhesive matrix may also include magnetizable particles for improving microcirculation.

The composite structure may include at least two layers of magnetizable particles capable of generating respective magnetic fields of different polarities.

In a particular embodiment, the composite structure has a support layer constituted by a non-woven cloth.

Still in a particular embodiment, the composite structure has two support layers defining its outside faces.

These two support layers can be of different roughnesses, porosities, or thicknesses so as to make two different types of application possible depending on which face is selected by the user.

In a particular embodiment, the composite structure has an impermeable support layer, e.g. in order to encourage diffusion of an active agent into the skin by preventing the composite structure from drying out.

Still in a particular embodiment, the composite structure has two juxtaposed or superposed adhesive matrices of compositions that are identical or different.

It can be advantageous to use an assembly comprising two or more adhesive matrices in order to obtain a desired combination of active agents rather than seeking to incorporate all of the active agents in the same adhesive matrix.

In particular, a given adhesive matrix can be manufactured in large quantity with one or more selected active agents and can then be assembled with one or more different adhesive matrices containing other active agents, so as to build up a range of composite structures having a variety of properties.

The weight per unit area of the matrix can lie in the range 10 grams per square meter (g/m²) to 100 g/m², for example.

In a particular embodiment, the composite structure comprises a stack of the following layers in this order: a first support structure; a first adhesive matrix containing at least one active agent; a second support layer; and a second adhesive matrix, which second matrix may optionally be covered in a removable protective film.

In another particular embodiment, the composite structure comprises a stack of the following layers in this order: a first support layer; a first adhesive matrix; a second adhesive matrix; and a second support layer.

In yet another particular embodiment, the composite structure comprises a stack of the following layers in this order: a first support layer; a first adhesive matrix containing at least one active agent; a second support layer; a second adhesive matrix containing at least one active agent; and a third support layer, the second support layer being impermeable and the first and third support layers being permeable, the first and second adhesive matrices containing different active agents.

Such composite structures are advantageously made by coating each support layer separately in adhesive matrix, and then assembling the various support layers once they have been coated.

In a particular embodiment, the composite structure has a first adhesive matrix comprising two juxtaposed regions containing different active agents. The support structure can further include a second adhesive matrix comprising two juxtaposed regions containing different active agents, possibly active agents other than those contained in the first adhesive matrix.

It is thus easy to multiply the combinations of active agents within a single composite structure.

During manufacture, each support layer coated in adhesive matrix is relatively easy to handle.

The composite structure of the invention can be made so as to constitute a patch for leaving on the skin for a predetermined length of time.

The composite structure of the invention can also be made in such a manner as to constitute a disk or a towelette for cleaning or treatment purposes.

The composite structure of the invention can also be made so as to constitute a towelette for treating the hair, e.g. for placing around a hair-curler.

The invention also provides a method of manufacturing a composite structure, the method comprising the following steps:

coating an adhesive matrix based on a permanent adhesive onto a support layer, said adhesive matrix containing at least one active agent and optionally a filler, the nature and the quantity of the active agent(s) and/or of the filler being selected to enable the active agent(s) to be released when the composite structure is wetted by a solvent; and

assembling together the support layer coated in this way in the adhesive matrix with a second support layer such that the adhesive matrix is sandwiched between the two support layers which are permanently bonded together by the matrix.

By way of example, the second support layer is coated on one face in a second adhesive matrix, in which case the two adhesive matrices can be stuck together.

It is advantageous to unite two adhesive matrices, even if they are identical in composition, since that makes it possible to assemble two different support layers together, e.g. to provide two outside faces that provide specific application characteristics.

It will be understood that the invention makes it easy to manufacture support layers independently and in large quantity, each of which is coated in an adhesive matrix containing one or more predetermined active agents, and subsequently to make up particular combinations of active agents depending on the use for which the composite structure is intended, merely by selecting appropriate support layers and assembling them together.

In particular, it is easy in this manner to make up a composite structure having two support layers and two adhesive matrices containing active agents that need to be stored separately.

The invention also provides a pile of composite structures, wherein the pile comprises at least two composite structures as defined above, each composite structure having at least one adhesive matrix disposed between two support layers, one of the support layers having an adhesive face in contact with the adjacent adhesive structure and said support layer constituting an extension enabling a user to take hold of the pile.

### BRIEF DESCRIPTION OF THE DRAWINGS

The invention will be better understood on reading the following detailed description of non-limiting embodiments, and on examining the accompanying drawings, in which:

FIGS. 1 to 10 are diagrammatic section views of various composite structures made in accordance with the invention;

FIG. 11 shows an implementation of the method of the invention;

FIG. 12 shows a cleaning disk;

FIG. 13 shows a patch for outlining the eye;

FIG. 14 is a plan view of an adhesive matrix comprising two adjacent regions containing different active agents;

FIG. 15 is a section through a variant embodiment of a composite structure; and

FIG. 16 shows a pile of composite structures.

### MORE DETAILED DESCRIPTION

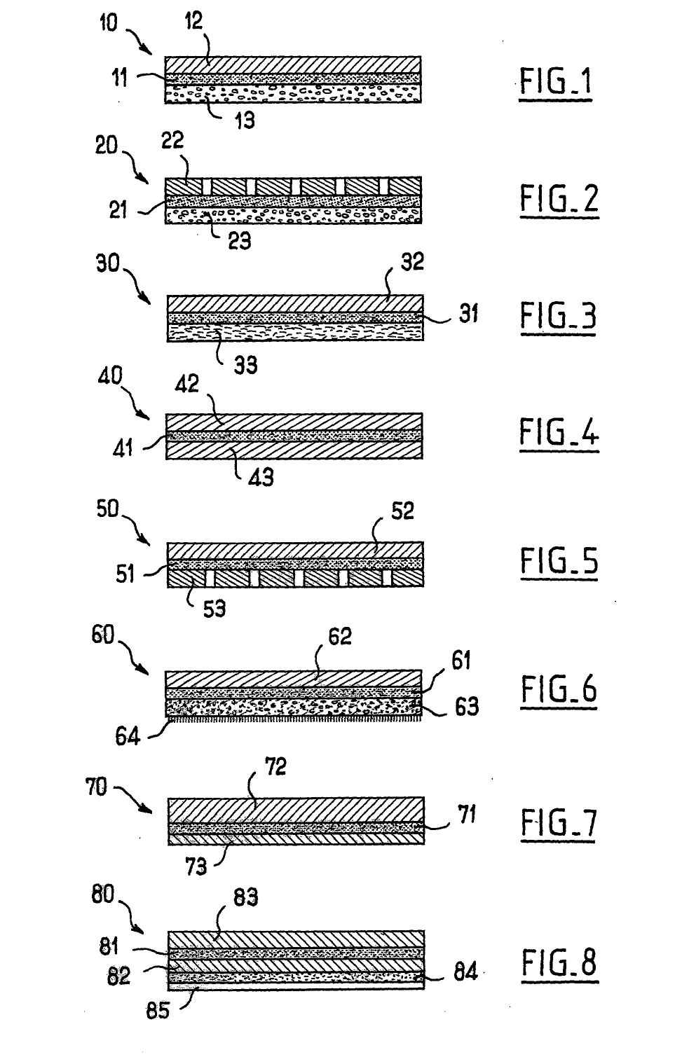

FIG. 1 shows a composite structure 10 constituting a first implementation of the invention.

This composite structure 10 comprises a layer of adhesive matrix 11 sandwiched between two support layers 12 and 13.

At least one of the support layers is permeable to a solvent, which in this case is constituted by water.

The adhesive matrix 11 provides a permanent adhesive basis that is not soluble in said solvent, thus making it possible to ensure that the two support layers 12 and 13 remain together even when the composite structure 10 is wetted.

In the example described, the adhesive matrix 11 contains at least one water-soluble active agent for cleaning, makeup, or care of the skin or the hair, and a filler enabling it to release the active agent(s) it contains when the composite structure 10 is impregnated with water or lotion.

To make the support layers 12 and 13, it is possible in particular to use a textile film, a non-woven cloth, or a cellular material such as a foam.

One of the support layers 12 or 13 can also be made using an impermeable film or a metal foil so as to make the composite structure waterproof.

The support layers 12 and 13 can be intrinsically hydrophilic or hydrophobic due to the nature of the material from which they are made, or they can be treated so as to make them hydrophilic or hydrophobic.

The support layers 12 and 13 can be of different thicknesses.

The adhesive matrix 11 can be based on vinyl compounds, on polyvinyl alcohol (PVA) or polyvinyl pyrrolidone (PVP), on pseudo-latexes such as acrylic polymers, on polyurethanes, or on latex elastomers, this list not being limiting.

The selected adhesive can be revisable (as applies to PVA or PVP for example) or not (as applies to acrylics, vinyl compounds, polyurethanes, and latex elastomers, for example).

The adhesive matrix 11 includes a filler capable of enabling it to absorb water so that it loses its cohesion and so that the hydrosoluble active agent(s) it contains is/are released when the composite structure 10 is wetted.

The filler can be constituted by particles of a water absorber, such as polyacrylate, for example.

In general, the adhesive matrix 11 can have incorporated therein 0.01% to 50% active agents selected, for example, from the following list: vitamin C, vitamin A, vitamin F, laponite, glycerin, wetting agents, collagen, salicylic acid, aromatic essential oils, coloring agents, caffeine.

It is preferable for the adhesive matrix also to have incorporated therein a powder filler of an inert material, e.g. ORGASOL.

A cleaning disk has been made having the structure shown in FIG. 1.

In the cleaning disk, the support layer 12 was constituted by a non-woven cloth treated to be hydrophilic, based essentially on viscose and having a small percentage of polypropylene. The adhesive matrix 11 was constituted by a permanent adhesive based on polyurethane and comprising by weight: 10% polyacrylate, 10% ORGASOL, and 0.5% non-anionic wetting agent, 0.2% mint crystals, and 0.5% mint essence. The support layer 13 was constituted by a polyurethane sponge.

The user can use the face defined by the sponge 13 for in-depth cleaning of the skin and can use the face defined by the non-woven cloth 12 to wipe the skin, for example.

FIG. 2 shows a composite structure 20 comprising an adhesive matrix 21 sandwiched between two support layers 22 and 23.

The structure shown in FIG. 2 differs from that shown in FIG. 1 by the fact that the support layer 22 has perforations.

By way of example, a cleaning disk has been made having structure as shown in FIG. 2, the support layer 22 being constituted by a hydrophilic perforated non-woven cloth made of polypropylene and viscose fibers with a weight per unit area of 10 g/m², the adhesive matrix 21 being constituted by a vinyl-based permanent adhesive including 15% ORGASOL, 5% laponite, 9% polyacrylate, 1.5% salicylic acid, and 0.5% retinyl palmitate, and the support layer 23 being constituted by a polyurethane sponge.

In the embodiment of FIG. 3, the composite structure 30 comprises an adhesive matrix sandwiched between two support layers 32 and 33.

The support layer 32 and the adhesive matrix 31 are identical respectively to the support layer 12 and to the adhesive matrix 11. The support layer 33 is constituted by a felt.

The composite structure 40 of the embodiment shown in FIG. 4 comprises an adhesive matrix 41 sandwiched between two support layers 42 and 43 respectively constituted by a polyethylene film having a thickness of 40 micrometers (μm) and by a hydrophilic non-woven cloth with a weight per surface area of 40 g/m², made up of a mixture of polypropylene and viscose fibers.

The adhesive matrix 41 is a matrix based on an acrylic permanent adhesive having 15% ORGASOL, 10% polyacrylate, 5% vitamin C, 15% glycerin, and 0.5% essence of orange.

The composite structure 40 is advantageously used for making a patch for applying to the skin for a predetermined length of time, e.g. lying in the range 5 minutes (min) to 20 min, in order to relax it, to soften it, and to give it tone.

The support layer 42 is waterproof and prevents the patch from drying out quickly on the skin.

The composite structure 50 shown in FIG. 5 comprises a support layer 52 and an adhesive matrix 51 that are respectively identical to the support layer 42 and to the adhesive matrix 41 in the preceding embodiment, while in this embodiment, the support layer 43 is replaced by a support layer 53 constituted by a perforated non-woven cloth.

The perforations encourage the active agents contained in the adhesive matrix 51 to diffuse towards the skin.

It should be observed that the perforations made in the support layer 53 can be sufficiently small to prevent the adhesive matrix from coming directly into contact with the skin, so as to ensure that the composite structure 50 does not stick to the skin.

The composite structure 60 of the embodiment of FIG. 6 has support layers 62 and 63 that are respectively identical to the support layer 12 of the embodiment of FIG. 1 and to the support layer 23 of the embodiment of FIG. 2, united by an adhesive matrix 61 identical to one of those described above. The support layer 63 is coated on its outside face by flocking 64.

FIG. 7 shows a composite structure 70 comprising an adhesive matrix 71 sandwiched between support layers 72 and 73 of different thicknesses.

By way of example, these support layers are constituted by non-woven cloths of different textures, one soft and the other rougher.

At the moment of use, the user can thus choose between two types of surface, e.g. depending on whether impurities on the surface of the skin are to be cleansed or whether mere superficial cleaning is to be performed.

The present invention is particularly advantageous in that it enables a plurality of adhesive matrices to be stacked directly or indirectly, thereby building up a wide variety of combinations of active agents and/or support layers having different properties.

The adhesive matrices can be assembled together in various ways, for example they can be stuck to each other or they can be individually sandwiched between support layers.

By way of example, FIG. 8 shows a composite structure 80 having a first adhesive matrix 81 sandwiched between two support layers 82 and 83.

The support layer 82 is coated on its face facing away from the adhesive matrix 81 in a second adhesive matrix 84, and this adhesive matrix is protected prior to use by a removable protective film 85.

The composite structure 80 can be used in various ways.

In particular, it can be applied to and left on skin that is wetted with water or lotion, like a patch, once the protective film 85 has been removed.

The adhesion of the adhesive matrix 84 on the skin can be used, where appropriate, to cleanse impurities or blackheads, which remain stuck to the adhesive matrix 84 when the composite structure 80 is removed.

The adhesive matrix 84 can have the same composition as one of the adhesive matrices described above, i.e. it can include at least one active agent and it can be arranged so as to release the active agent once it comes into contact with the skin.

The adhesive matrix 84 can equally well comprise permanent adhesive only, with the active agent(s) then being contained in releasable manner in the adhesive matrix 81.

Preferably, both of the adhesive matrices 81 and 84 contain active agents.

The composite structure can also be used after it has been soaked in water without removing the protective film 85, to be applied to the skin via its face defined by the support layer 83, e.g. to clean the skin.

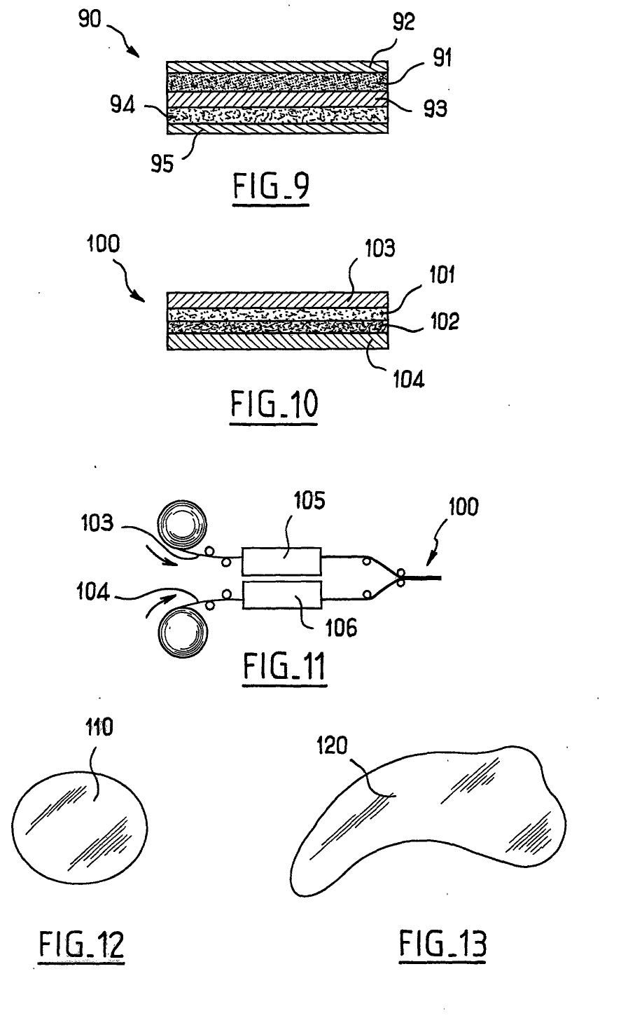

In the embodiment of FIG. 9, the composite structure 90 has a first adhesive matrix 91 sandwiched between two support layers 92 and 93 and a second adhesive matrix 94 sandwiched between the support layer 93 and another support layer 95.

The support layer 93 which is sandwiched between the adhesive matrices 91 and 94 can be made of a material that is selected to be water permeable or water impermeable.

When the selected material is impermeable to water, the support layers 92 and 95 are permeable to water, thereby enabling them to reach the adhesive matrices 91 and 94 when the composite structure 90 is wetted.

The adhesive matrices 91 and 94 then advantageously have different active agents, and the user can thus treat the skin in different manners depending on whether the support layer 92 or the support layer 95 is placed against the skin.

Only those active agents contained in the adhesive matrix 91 diffuse into the support layer 92, given the existence of the impermeable barrier constituted by the support layer 93.

Similarly, only those active agents that are contained in the adhesive matrix 94 diffuse into the support layer 95.

FIG. 10 shows a composite structure 100 having two adhesive matrices 101 and 102 that are stuck together, being sandwiched between two support layers 103 and 104.

One of the support layers 103 and 104 can be waterproof.

In this example, the adhesive matrices 101 and 102 have different active agents, e.g. active agents unsuitable for being packaged together.

As shown in FIG. 11, to make the composite structure 100, two support layers 103 and 104 are used each having a respective adhesive matrix 101 and 102 deposited thereon in a respective conventional coating station 105 or 106.

The adhesive matrices 101 and 102 can contain solvents during manufacture in order to facilitate the coating operation.

These solvents are volatile and for elimination from the final composite structure.

The support layers 103 and 104 coated in this way in their respective adhesive matrices are then stuck together to form the composite structure 100.

A cleaning disk has been made having the structure shown in FIG. 10, the support layer 103 being constituted by a non-woven cloth, the adhesive matrix 101 being based on a permanent polyacrylic adhesion containing 2% citric acid, 20% glycerin, 15% ORGASOL, and 1% caffeine, the adhesive matrix 102 being based on a permanent polyvinyl adhesive containing 0.2% vitamin A, 6% bicarbonate, 0.2% kojic acid, and 0.5% of a cationic wetting agent, and the support layer 104 being constituted by a non-woven cloth.

In use, the bicarbonate reacts with the citric acid so as to form a foam.

It will be understood that the invention makes it possible to make a plurality of support layers independently, each of which is impregnated with an adhesive matrix containing predetermined active agents, and to assemble together the support layers coated in this way so as to obtain a desired combination of active agents.

The composite structures described above can be cut into various shapes, depending on the intended application.

By way of example, FIG. 12 shows a cleaning disk 110 and FIG. 13 shows a patch 120 for outlining the eyes.

An adhesive matrix having one of the above-described composite structures can have two juxtaposed regions containing different active agents.

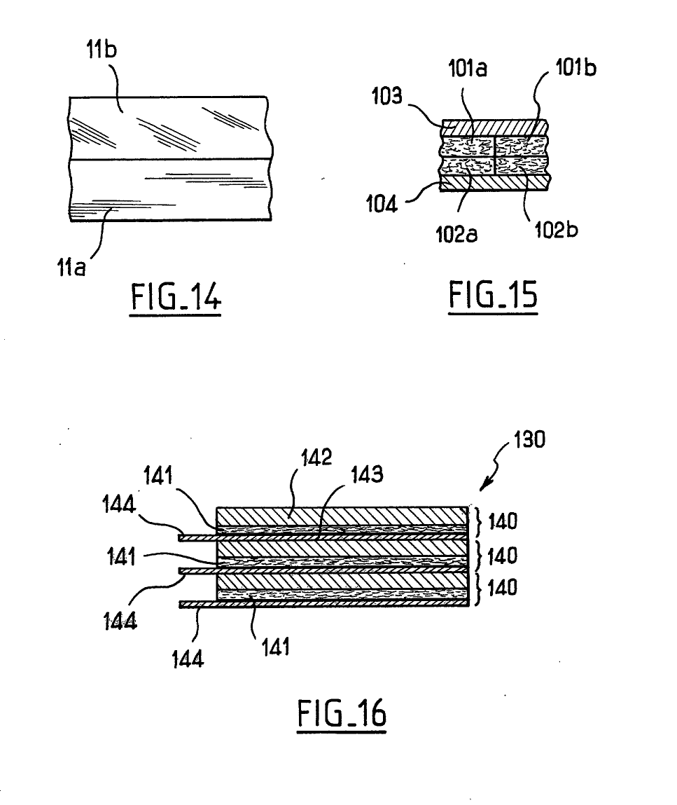

By way of example, FIG. 14 shows an adhesive matrix having two regions 11a and 11b containing different active agents and replacing the adhesive matrix 11 as described above.

This configuration can be used in particular when it is necessary for a plurality of active agents that need to be stored separately to be packaged within a single adhesive matrix.

The configuration of FIG. 14 can also be used to increase the number of active agents that are stored separately in a composite structure of the kind shown in FIG. 10, for example.

FIG. 15 shows a composite structure comprising two adhesive matrices that are stuck together, each matrix comprising respective juxtaposed regions 101a & 101b and 102a & 102b that contain different active agents.

FIG. 16 shows a pile 130 of composite structures 140, each composite structure 140 having an adhesive matrix 141 that can be any one of the adhesive matrices described above, the adhesive matrix 141 being sandwiched between two support layers 142 and 143.

The support layer 143 has a bottom face that comes into contact with the underlying composite structure 140 and presents the feature of including an extension 144 that constitutes a tongue which the user can take hold of.

The support layer 143 preferably receives adhesive treatment on its face that comes into contact with the underlying composite structure 140 so as to enable the various composite structures 140 in the pile to be held in place.

Naturally, the invention is not limited to the embodiments described above.

In particular, it is possible to use other combinations of support layers and adhesive matrices.

The adhesive matrix can also be used as a reservoir of active agents and the composite structure can be wetted several times over.

It is possible to use a solvent other than water for wetting the composite structure, providing it is compatible with external use on a user.

1. A composite structure such as a patch, disk, or towelette used in particular for treating, making up, or cleaning the skin or the hair, the structure comprising at least one adhesive matrix based on a permanent adhesive and present between two support layers, at least one of which is permeable to a solvent, the two support layers being permanently bonded to the adhesive matrix, the adhesive matrix containing at least one active agent soluble in said solvent and possibly a filler, the nature and the quantity of the active agent(s) and/or the filler being selected to make it possible, when the composite structure is wetted by the solvent, for the active agent(s) to be released and to diffuse towards the surface to be treated.

2. A composite structure according to claim 1, wherein the adhesive matrix contains one or more active agents soluble in the solvent and/or capable of swelling on coming into contact with said solvent, and in sufficient quantity for the matrix to lose its cohesion on contact with the solvent and to release the active agent(s).

3. A composite structure according to claim 1, wherein the adhesive matrix contains a filler of one or more compounds capable of swelling on contact with the solvent, in sufficient quantity for the matrix to lose its cohesion on contact with the solvent and to release the active agent(s).

4. A composite structure according to claim 1, wherein the adhesive matrix contains a filler of one or more substantially inert compounds in sufficient quantity for the matrix to lose its cohesion on contact with the solvent and to release the active agent(s).

5. A composite structure according to claim 1, wherein said solvent is water.

6. A composite structure according to claim 1, wherein the matrix contains at least one water-soluble active agent.

7. A composite structure according to claim 1, wherein the matrix includes one or more moisture-absorbing compounds.

8. A composite structure according to claim 1, wherein the adhesive matrix contains 0.2% to 60% by weight of a moisture-absorbing compound, and preferably 0.5% to 40%.

9. A composite structure according to claim 1, wherein the adhesive matrix includes at least one moisture-absorbing compound selected from the following list: polyacrylates; silicas; cotton fibers; starches; alginates; calcium carbonates; magnesium; viscose; cellulose; and freeze-dried substances.

10. A composite structure according to claim 1, wherein the adhesive matrix has one or more substantially inert substances such as microbeads or a powder of an inert compound, in particular the polyamide powder known under the name ORGASOL.

11. A composite structure according to claim 1, wherein the active agent(s) is/are selected from the following list: vitamin C; vitamin A; vitamin F; glycerin; laponite; wetting agents; collagen; salicylic acid; tio acid; caffeine; aromatic essential oils; coloring agents; anti-oxidants; free radical scavengers; moisturizers; depigmenting agents; liporegulators; anti-acne agents; antidandruff agents; anti-aging agents; softeners; antiwrinkle agents; keratolitic agents; anti-inflammatory agents; fresheners; healing agents; vascular protectors; antibacterial agents; antifungal agents; antiperspirants; deodorants; skin conditioners; anesthetics; immunomodulators; and nourishing agents.

12. A composite structure according to claim 1, wherein the adhesive matrix includes magnetizable particles.

13. A composite structure according to claim 12, including at least two layers of magnetizable particles capable of generating respective magnetic fields of different polarities.

14. A composite structure according to claim 1, wherein the adhesive matrix is based on a permanent adhesive selected from the following list: adhesive based on vinyl; on PVA or PVP; on pseudo-latex; on acrylic polymers; on polyurethanes; and on latex elastomers.

15. A composite structure according to claim 1, including a support layer constituted by a non-woven cloth.

16. A composite structure according to claim 1, having two outer support layers that are permeable to the solvent.

17. A composite structure according to claim 16, wherein said support layers have different roughnesses, porosities, or thicknesses so as to enable two different types of application to be performed depending on which face is selected by the user.

18. A composite structure according to claim 1, including an impermeable support layer.

19. A composite structure according to claim 1, including two juxtaposed or superposed adhesive matrices of compositions that are identical or different.

20. A composite structure according to claim 19, wherein said adhesive matrices are stuck to each other and include different active agents.

21. A composite structure according to claim 1, comprising a stack of the following layers in this order: a first support layer; a first adhesive matrix containing at least one active agent; a second support layer; and a second adhesive matrix essentially covered by a removable protective film.

22. A composite structure according to claim 1, comprising a stack of the following layers in this order: a first support layer; a first adhesive matrix containing at least one active agent; a second support layer; a second adhesive matrix containing at least one active agent; and a third support layer, the second support layer being impermeable and the first and third support layers being permeable, the first and second adhesive matrices containing different active agents.

23. A composite structure according to claim 1, comprising a stack of the following layers in this order: a first support layer; a first adhesive matrix; a second adhesive matrix; and a second support layer.

24. A composite structure according to claim 21, wherein the first and second adhesive matrices have respective active agents that need to be stored separately.

25. A composite structure according to claim 1, including an adhesive matrix comprising two juxtaposed regions containing different active agents.

26. A composite structure according to claim 25, further including a second adhesive matrix comprising two juxtaposed regions containing different active agents, optionally constituted by active agents other than those of the first adhesive matrix.

27. A method of manufacturing a composite structure such as a patch, a disk, or a towelette for cleaning or treatment purposes, the method comprising the following steps:
coating an adhesive matrix based on a permanent adhesive onto a support layer, said adhesive matrix containing at least one active agent and optionally a filler, the nature and the quantity of the active agent(s) and/or of the filler being selected to enable the active agent(s) to be released when the composite structure is wetted by a solvent; and
assembling together the support layer coated in this way in the adhesive matrix with a second support layer such that the adhesive matrix is sandwiched between the two support layers which are permanently bonded together by the matrix.

28. A method according to claim 27, wherein the second support layer is coated on one face in a second adhesive matrix.

29. A method according to claim 28, wherein the two adhesive matrices are stuck together.

30. A method according to claim 27, wherein a large quantity of support layers coated in adhesive matrices containing predetermined active agents are manufactured separately, and wherein the various support layers coated in this way are assembled together to make up a range of composite structures presenting different combinations of active agents.

31. The use of a composite structure as defined in claim 1 as a patch applied to the skin for a predetermined length of time.

32. The use of a composite structure as defined in claim 1, for cleaning the skin.

33. The use of a composite structure as defined in claim 1, for treating the hair.

34. The use of a composite structure as defined in claim 1, the adhesive matrix being used as an active agent reservoir and the composite structure being wetted several times over.

35. A pile of composite structures, comprising at least two composite structures as defined in claim 1, each composite structure having at least one adhesive matrix disposed between two support layers, one of its support layers having an adhesive face in contact with the underling adhesive structure and having an extension enabling it to be taken hold of by a user.
